(19) United States
(12) Patent Application Publication
(10) Pub. No.: US 2025/0303051 A1
(43) Pub. Date: Oct. 2, 2025

(54) Device for purifying adipose tissue

(71) Applicant: NEOSYAD, AIX-EN-PROVENCE (FR)

(72) Inventors: Régis ROCHE, LAMBESC (FR); François CABAUD, EGUILLES (FR); Xavier NELISSEN, VILLERS SAINT SIMEON (BE)

(21) Appl. No.: 18/863,686

(22) PCT Filed: Apr. 28, 2023
(86) PCT No.: PCT/FR2023/000067

§ 371 Date: Nov. 7, 2024

(30) Foreign Application Priority Data
May 9, 2022 (FR) FR2204370

(51) Int. Cl. A61M 1/00; A61K 35/35

(52) U.S. Cl. CPC A61M 1/892; A61K 35/35; A61M 1/79; A61M 1/895; A61M 2202/08

(57) ABSTRACT

A device for purifying adipose tissue may comprise a chamber, a flexible filter delimiting a treatment volume and being able to retain the adipose tissue and to filter a liquid medium, the filter being present in the chamber and extending along a main axis between a lower end and an upper end, a lower attachment element secured to the lower end of the filter and an upper attachment element secured to the upper end of the filter, a discharge duct extending into the treatment volume between a lower portion provided with the inlet orifice and an upper portion in communication with the outside of the device, and a drive system configured to take a purification configuration in which the lower and upper attachment elements are rotated jointly around the main axis and configured to take a discharge configuration.

### TECHNICAL FIELD

The present invention concerns a device for purifying adipose tissue and an associated purification method. The invention especially finds application for autologous adipose tissue transplantation procedures (“lipofilling”) with aesthetic and reconstructive aims. In particular, the invention finds a use for breast surgery operations; however, the implementation of the invention is not limited to this application.

### PRIOR ART

Autologous adipose tissue transplantation procedures are used in surgery, especially breast surgery, to shape the breast to a more natural appearance after deep inferior epigastric perforator (DIEP), latissimus dorsi flap reconstruction, or after placing a breast implant. These operations involve taking fat from a patient in a donor area and then reintroducing it into the area of interest of the patient's body. These operations currently do not have a reproducible rendering and may require several interventions due to the resorption of the introduced material (loss of volume encountered after the operation). In addition, the fat is prepared for its reintroduction by an operator, surgeon or surgical assistant, which generates a relatively high cost of implementing the operation and a risk of error or loss of reproducibility. It is therefore desirable to propose a relatively simple technique which makes existing operations more reliable.

The document US Patent No. 2020/0054824 sought to propose an improvement by implementing a closed loop system in which the fat is harvested to be prepared with a view to its reintroduction by being mixed with a washing liquid in a gravity filtration device marketed under the commercial reference Revolve®, and then to be reintroduced into the patient's body using a centrifugal pump. However, it is possible to improve the quality of the reintroduced fat. US 2020/061259 is also known, which discloses a device for treating fat cells taken from a patient and intended for a graft.

### DISCLOSURE OF THE INVENTION

The application concerns a device for purifying adipose tissue and discharging the purified tissue comprising:
a chamber,
a flexible filter delimiting a treatment volume and being able to retain the adipose tissue and to filter a liquid medium, the filter being present in the chamber and extending along a main axis between a lower end and an upper end in communication with the outside of the device,
a lower attachment element secured to a lower end of the filter and an upper attachment element secured to an upper end of the filter,
a discharge duct extending into the treatment volume between a lower portion provided with an inlet orifice and an upper portion in communication with the exterior of the device, and
a drive system configured to take a purification configuration in which the lower and upper attachment elements are rotated jointly around the main axis, and configured to take a discharge configuration in which one of the attachment elements is rotated around the main axis and in relative motion with regard to the other attachment element so as to wind the filter on itself.

The application proposes a device of relatively simple design making it possible to purify adipose tissue and discharge this purified adipose tissue for reintroduction into the patient's body. The device removes contaminants from adipose tissue, which may comprise at least one of oil, blood or water. A washing liquid may be added to the adipose tissue in the treatment volume to recover a fraction of the contaminants which is then filtered out by centrifugation of the contents of the treatment volume. The device is based on the fact that the ends of the filter are connected to separate attachment elements whose movement is controlled differently by the drive system depending on whether it is desired to purify the adipose tissue or to carry out the discharge. Thus, in the purification configuration, the lower and upper ends of the filter rotate together, which leads to centrifugation of the adipose tissue against the internal wall of the filter and discharging the washing liquid containing the contaminants allowing the adipose tissue to be purified. The liquid medium present in and around the adipose tissue is then effectively drained through the filter while the adipose tissue is retained in the treatment volume. A large quantity of liquid is thus eliminated, making it possible to obtain purified adipose tissue which, once reimplanted, has a much lower resorption rate than that obtained with the solutions of the prior art. In return, when the drive system is put into the discharge configuration, one end of the filter is rotated with relative movement with regard to the other end, which can be fixed by being no longer engaged with the drive system. This causes the filter to wind on itself from its upper end (area of least stress with no adipose tissue, or less adipose tissue than in the lower end) to its lower end. This winding applies discharge pressure that pushes purified adipose tissue into the discharge duct to allow it to be transferred outside the device for reintroduction into the patient's body.

In an exemplary embodiment, the device further comprises a rigid cage for retaining the filter, surrounding the filter and situated between it and the chamber, which is capable of being traversed by the liquid medium.

The retention cage makes it possible to retain the filter deformed under the effect of centrifugal force during purification in order to avoid any risk of it coming into contact with the chamber and of the drainage of the liquid medium being disturbed. This advantageously makes it possible to make the device more compact without hindering the quality of the purification.

In particular, the rigid retention cage may define the upper attachment element and may be configured to be rotated in the purification configuration.

Such a characteristic further simplifies the design of the device by reducing the number of elements necessary by a combination of the filter retention function and the filter attachment.

In an exemplary embodiment, said other attachment element is fixed in the discharge configuration.

In other words, in this case, in the discharge configuration, the upper attachment element is fixed while the lower attachment element rotates, or conversely the lower attachment element is fixed while the upper attachment element is rotary. Such a characteristic makes it possible to obtain a rapid winding of the filter on itself with a relatively simple drive system.

In an exemplary embodiment, the device further comprises a collapsible element to assist discharge located around the inlet orifice of the discharge duct, said collapsible element being configured to go from a deployed configuration in the purification configuration to a collapsed configuration in response to the winding of the filter in the discharge configuration.

Such a characteristic makes it easier to push purified adipose tissue into the discharge duct in order to maximize the output of the operation.

The application also concerns an assembly for purifying adipose tissue and introducing the purified adipose tissue into the body of a patient, comprising a device as described above, a discharge segment in communication with the upper portion of the discharge duct, and a surgical device comprising (i) a reservoir for receiving purified adipose tissue and in communication with the discharge segment, and (ii) an introduction element in communication with the reservoir and adapted to introduce the purified adipose tissue from the reservoir into the patient's body.

The application also concerns a method for purifying adipose tissue using a device as described above containing adipose tissue to be purified in the treatment volume, comprising:
centrifuging the adipose tissue by placing the drive system in the purification configuration, and
discharging the purified adipose tissue, obtained after centrifugation, by placing the drive system in the discharge configuration so as to wind the filter on itself and thus apply a discharge pressure to the purified adipose tissue allowing it to be transported outside the device through the discharge duct.

The application also concerns an element for filtering adipose tissue intended to be integrated into a device as described above, comprising:
the flexible filter delimiting the treatment volume and able to retain the adipose tissue and to filter a liquid medium, the filter extending along the main axis between the lower end and the upper end,
the lower attachment element being secured to the lower end of the filter, comprising a means of cooperation with the drive system to achieve rotation around the main axis, and
the discharge duct extending into the treatment volume between the lower portion provided with the inlet orifice and the upper portion in communication with the outside of the treatment volume.

Such an object is intended to be mounted to the other constituent elements of the device before carrying out the treatment of the adipose tissue. It is the disposable element that can be removed from the rest of the device and replaced after the purified adipose tissue has been removed. This filtration element is in a sterile state. In an exemplary embodiment, the filtration element further comprises a collapsible element to assist discharge located around the inlet orifice of the discharge duct, the collapsible element being configured to go from a deployed configuration in the purification configuration to the collapsed configuration in response to the winding of the filter in the discharge configuration.

### BRIEF DESCRIPTION OF THE DRAWINGS

FIG. 1 schematically and partially represents an example of a purification device.

FIG. 2 schematically and partially represents the device of FIG. 1 during the purification of adipose tissue.

FIG. 3 schematically and partially represents the device of FIG. 1 during the discharge of purified adipose tissue.

FIG. 4 schematically and partially represents a surgical device capable of allowing the reintroduction of purified adipose tissue into the patient's body, which can be used within the scope of the application.

FIG. 5 schematically and partially represents a variant of a filtration element equipped with a collapsible element to assist discharge in the deployed configuration.

FIG. 6 schematically and partially represents the filtration element of FIG. 5 with the collapsible element being moved to the collapsed configuration by the winding of the filter.

FIG. 7 schematically and partially represents the filtration element of FIG. 5 with the collapsible element in the collapsed configuration.

### DESCRIPTION OF THE EMBODIMENTS

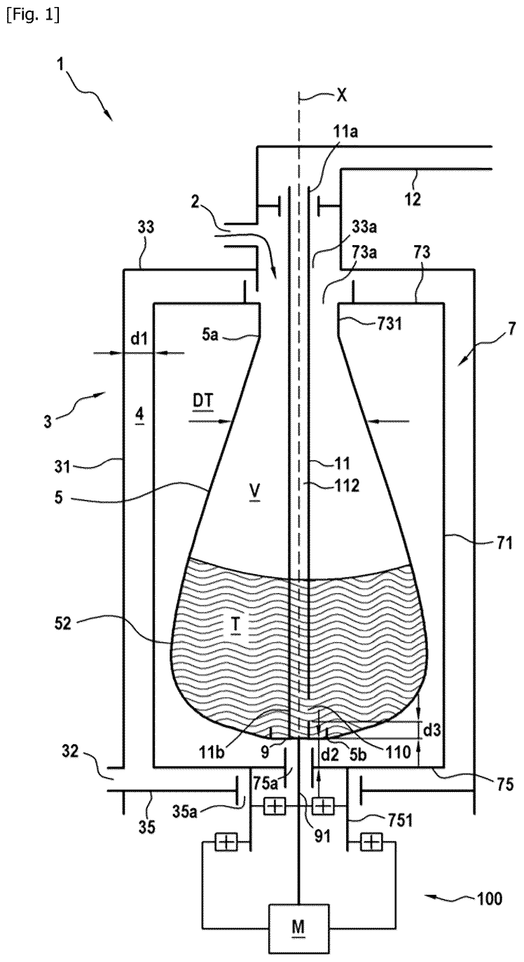

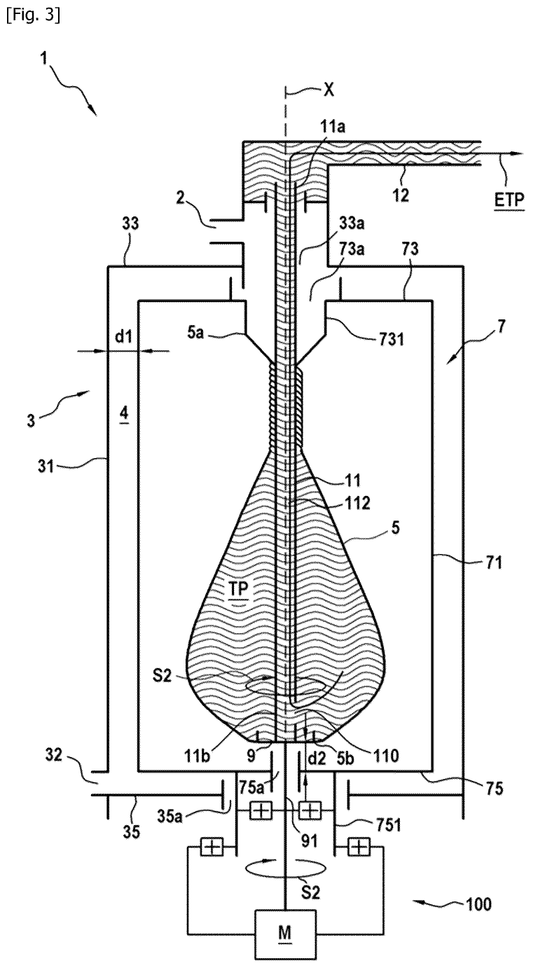

FIG. 1 represents a device 1 for purifying adipose tissue T taken from a patient's body. The device 1 comprises a chamber 3 defining the outer surface of the device 1. The chamber 3 may be formed of a material transparent to visible light, such as plexiglass, so that the contents of the chamber can be viewed by the surgeon during the operation. The chamber 3 comprises a cover 33 in communication with an inlet port 2 through which the adipose tissue T is introduced into the device 1. The port 2 may be located above the chamber, as shown. The chamber 3 may also have a suction port 32 whose function will be detailed below. The port 2 may be connected to a suction cannula (not shown) for removing the adipose tissue T from the patient's body, and the introduction of the adipose tissue T removed in the device 1 may be assisted by suction through the port 32 using a pumping device (not shown). FIG. 1 shows the device 1 being filled with adipose tissue T. In the example considered, the adipose tissue T passes successively through the port 2, a first opening 33a of the cover 33 and then a second opening 73a defined by a rigid retention cage 7 to be collected in a treatment volume V. The volume V is defined by a filter 5 which is present in the chamber 3 and situated inside the cage 7. Various solutions can be envisaged for holding the cage 7 in place in the chamber 3, for example by using rods (not shown) extending between the cover 33 and the lower edge 35 of the chamber 3. The filter 5 here forms a pocket receiving the adipose tissue T to carry out its purification. The filter 5 is able to retain the adipose tissue T but allows a liquid medium containing contaminants to be eliminated to pass through. The filter 5 may have a plurality of pores of a size adapted to this function. The pore size is chosen, in particular, to allow the passage of liquids such as oil, blood or physiological solution while retaining the adipose tissue T. In general, the pore size of the filter 5 can be less than or equal to 1.5 mm, for example less than or equal to 0.5 mm. This size can be comprised between 0.05 mm and 1.5 mm, for example, between 0.2 mm and 0.5 mm. The filter 5 can be made of a polymer material, for example polyester or polypropylene, but the person skilled in the art will recognize that other materials can be used. The filter 5 is made of flexible material, i.e., a material capable of twisting in order to discharge the purified adipose tissue outside the device 1, as will be described below in connection with FIG. 3.

The cage 7 surrounds the filter 5 to prevent it from coming into contact with the chamber 3, in particular with its side wall 31, during centrifugation. The presence of the cage 7 remains optional, especially for devices having a chamber of greater transverse dimension sufficient to guarantee a spacing between the filter and the chamber during centrifugation. The cage 7 is rigid, therefore does not deform under the effect of centrifugal force during purification so as to retain the filter 5. The cage 7 may be made of a metal or plastic material. The cage 7 comprises a lateral portion 71 having a plurality of orifices through which the contaminants to be discharged can pass. The cage 7 is separated from the chamber 3 by a non-zero distance d1 so as to define a space 4 for concentrating the centrifuged contaminants for their subsequent discharge by suction through the port 32. The lateral portion 71 is situated between an upper edge 73, defining the opening 73a mentioned above, and a lower edge 75 defining an opening 75a. The cage 7 extends along a main X axis corresponding to the height axis of the device. The cage 7 may or may not have a shape of revolution around the X axis, for example by having a substantially cylindrical shape. In the example illustrated, an upper attachment element 731 secured to an upper end 5a of the filter 5 is defined by the upper edge 73 of the cage 7. The element 731 can form, as illustrated, a fastening collar which is surrounded by the lateral portion 71 and connected to the end 5a. The element 731 is here traversed by the adipose tissue T introduced and emerges in the treatment volume V.

The filter 5 extends along the X axis between the upper end 5a and a lower end 5b. As indicated above, the upper end 5a is here connected to the cage 7, and more particularly to the fastening collar 731 which forms the upper fastening element. Various solutions can be used to connect the end 5a to the element 731 such as gluing, crimping or clipping. The lower end 5b is secured to a lower attachment element 9, here in the form of a support transverse to the X axis, which is distinct from the cage 7. Here, the element 9 is situated inside the cage 7 and is surrounded by the lateral portion 71. The element 9 may be distant from the cage 7 by being spaced from its lower edge 75 by a non-zero distance d2. The element 9 can delimit the bottom of the treatment volume V and of the filter 5. Various solutions can be used to connect the end 5b to the element 9 such as gluing, crimping or clipping. The element 9 is connected to a drive system 100 by means of an axle 91 capable of cooperating with this system 100. The element 9 comprises a means of cooperation with the axle 91. The axle 91 extends along the X axis and passes through the opening 75a to the outside of the chamber 3, passing through the opening 35a defined by the lower edge 35 of the chamber 3. The axle 91 cooperating with the system 100 and the element 9 makes it possible to rotate the element 9 around the X axis. The cage 7 is also connected to the system 100 by means of its lower edge 75 which defines one or more drive portions 751 extending outside the chamber 3. The drive portions 751 are adapted to cooperate with the system 100 so as to rotate the cage 7 around the X axis, and in particular to rotate the element 731.

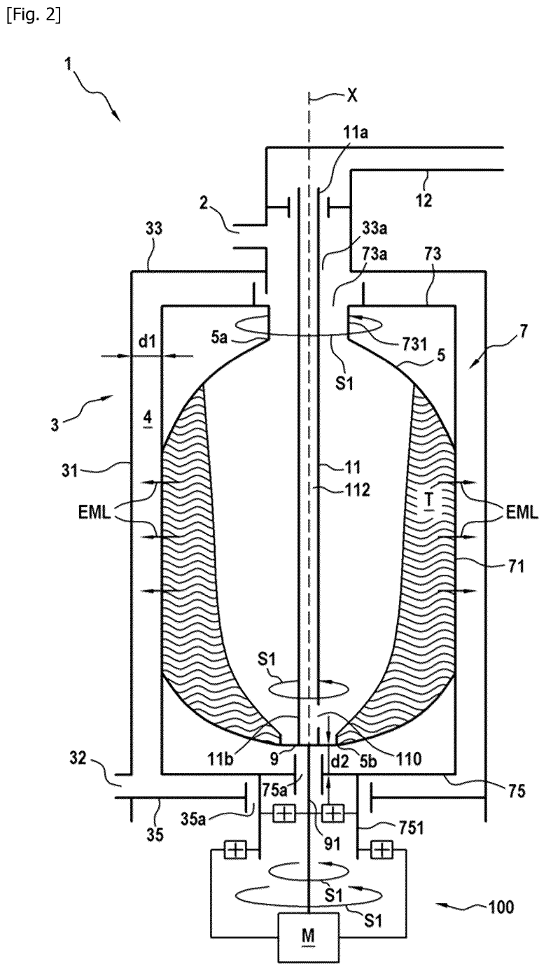

The system 100 has a first configuration, called the purification configuration, in which it cooperates with the element 9 and the element 731 so as to drive them in joint rotation around the X axis. Thus, the ends 5a and 5b of the filter rotate together, which allows centrifugation of the adipose tissue T as illustrated in FIG. 2. The same rotation speed can be imposed on the elements 9 and 731 in the purification configuration. When the purification is complete, the system 100 can be put into a second configuration, called the discharge configuration, in which there is a relative movement of one element 9 with respect to the other element 731 which will produce a winding of the filter 5 on itself. In the example illustrated, the element 9 cooperates with the system 100 but not with the element 731 in the discharge configuration. Thus, the element 9 is driven in rotation around the X axis while the element 731 remains fixed, which leads to a winding of the filter 5 as illustrated in FIG. 3, resulting in the discharge of the purified adipose tissue TP through a discharge duct 11. The discharge duct 11 extends inside the filter 5 along the main X axis between a lower portion 11b, which is here connected to the lower attachment element 9, and an upper portion 11a in communication with the outside of the device 1. The portion 11a opens out above the cover 33 into a discharge segment 12 connected to the cover 33 and intended to transfer the purified adipose tissue to a surgical device capable of reintroducing it into the patient's body, possibly with the assistance of a pump to facilitate this transfer. Various solutions can be used to connect the lower portion 11b to the element 9 such as gluing, crimping or clipping. The portion 11b is provided with one or more inlet orifices 110 placing the volume V in communication with the inside 112 of the duct 11 so as to discharge the purified adipose tissue outside the device 1 due to the pressure applied by the filter 5 when it is wound. The orifice or orifices 110 may be in the vicinity of the element 9, for example at a distance d3, measured along the X axis, less than or equal to 50 mm from the element. The distance d3 may be substantially equal to 10 mm or less than 10 mm. Winding the filter for the discharge of purified adipose tissue TP has been described, obtained by rotating element 9 while keeping the element 731 immobile, but it will be recognized that it does not exceed the scope of the application if the reverse is carried out with a fixed element 9 and an rotated element 731, or by rotating the lower end 5b and upper end 5a with non-zero and different rotation speeds, or even rotating in the opposite directions. The system 100 may, for example, comprise a bidirectional clutch system known per se, capable of driving the elements 9 and 731 jointly in a first direction of rotation S1 in the purification configuration, and of driving only one of the elements 9 or 731 in a second direction of rotation S2 in the discharge configuration. The system 100 is here connected to an electric motor M which may form an integral part of the device or be present in a plate separate from the device on which the device 1 is intended to be positioned. As a variant, a manually driven device 1 could be used.

Various details of the structure and operation of an example of a device 1 have just been described. The following reviews the various operating steps in more detail: filling volume V with adipose tissue T to be purified, purifying this adipose tissue and discharging the adipose tissue thus purified for reintroduction into the patient's body.

The device 1 is, of course, in a sterile state before its use. As illustrated in FIG. 1, the adipose tissue T falls by gravity from the port 2 into the volume V. The flexible filter 5 deforms as the adipose tissue T is filled so as to form an area 52 of greater transverse dimension DT and may define a neck at the upper end 5a of the filter 5. The transverse dimension DT of the filter 5 containing the adipose tissue T, taken perpendicularly to the main X axis, may be strictly decreasing from the zone 52 toward the end 5a. FIG. 2 illustrates the purification of adipose tissue T by centrifugation in which the system 100 drives the cage 7 and the element 9 in joint rotation, as indicated above. The liquid medium is discharged through the filter 5 by application of centrifugal force (arrows EML) and the adipose tissue is retained in the volume V. There is no winding of the filter 5 in the purification configuration. As illustrated in FIG. 2, the flexible filter 5 deforms during centrifugation, the cage 7 does not deform and retains the filter 5 to avoid contact with the chamber 3, in this case with its side wall 31. When the filter 5 is rotated in the purification configuration, the adipose tissue and the contaminants can undergo an acceleration greater than or equal to 8 G, for example greater than or equal to 10 G or greater than or equal to 12 G. This acceleration measured in G corresponds to the ratio between the acceleration undergone by the material and the acceleration of Earth's gravity, which is approximately 9.81 m²/s. The acceleration experienced by the material corresponds to the ratio of the centrifugal force applied to the mass of the material concerned. The centrifugal force applied is equal to m*ω²*R where m is the mass of the object in question, ω is the angular velocity of the filter 5 expressed in rad/s and R is the distance from the X axis of rotation to the center of gravity of the object in question. During the rotation of the filter 5 in the purification configuration, the adipose tissue and the contaminants can undergo an acceleration less than or equal to 40 G, for example less than or equal to 30 G, or less than or equal to 25 G or less than or equal to 20 G. This acceleration can be comprised between 8 G and 40 G or between 8 G and 30 G or between 8 G and 25 G or between 8 G and 20 G. This acceleration can be comprised between 10 G and 40 G or between 10 G and 30 G or between 10 G and 25 G or between 10 G and 20 G. This acceleration can be comprised between 12 G and 40 G or between 12 G and 30 G or between 12 G and 25 G or between 12 G and 20 G. It should be noted that it is possible to rotate the filter 5 intermittently during purification, i.e., by successively imposing at least a first phase of rotating the filter 5, a phase of interrupting this rotation where the filter 5 is immobilized, and a second phase of rotating the filter 5. It is thus possible to alternate phases of rotation of the filter 5 and phases of interruption of this rotation during purification.

Purification can be carried out with or without the addition of a washing liquid, for example in the form of a physiological solution. It will be noted that purification can be carried out while the adipose tissue T is being introduced into the volume V and can possibly be continued once this introduction is complete. The contaminants are drawn through the port 32 to be discharged outside the device 1.

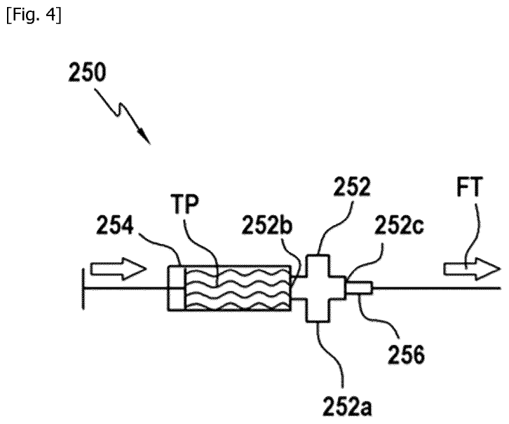

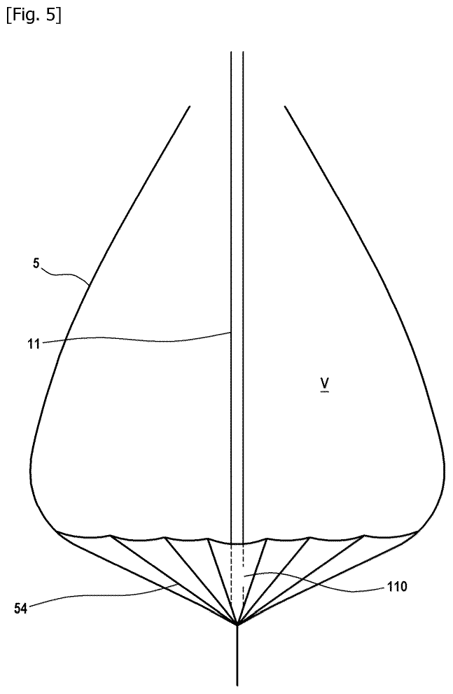

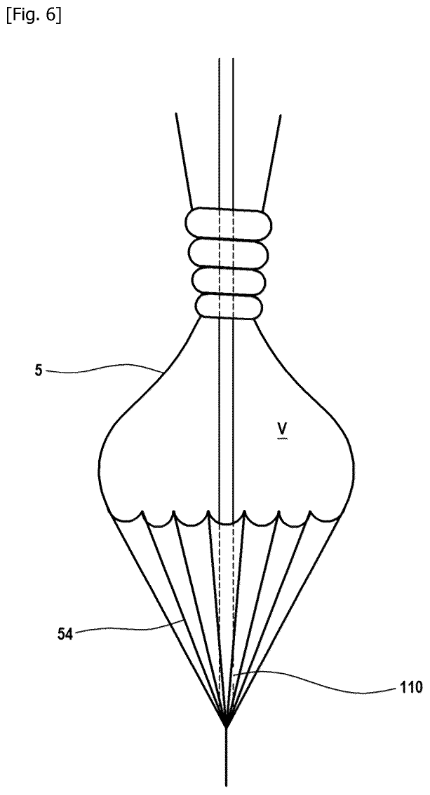

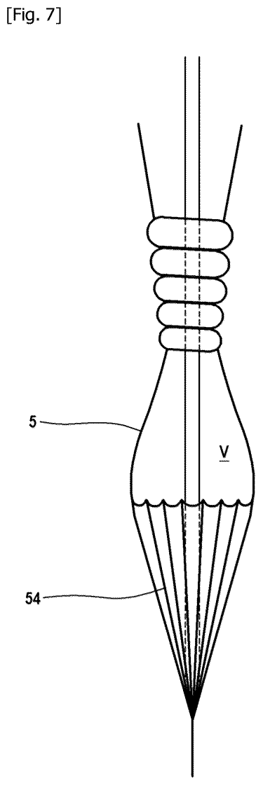

Once purification is complete, system 100 is placed in the discharge configuration to transfer purified adipose tissue TP out of the device for reintroduction into the patient. As indicated above and schematically in FIG. 3, the discharge configuration results in a winding of the filter 5 on itself which produces a pressure pushing the purified adipose tissue TP through the orifice 110 and the interior 112 of the duct 11 to reach the discharge segment 12. The purified adipose tissue TP is then transferred into a surgical device 250 that allows it to be reintroduced into the patient (represented in FIG. 4), such as, for example, a device marketed under the reference LipoGrafter® by the company MTFBiologics. The torque given to the motor makes it possible to adjust the pressure exerted during winding and therefore to calibrate the desired flow rate. As indicated above, a pump may or may not be disposed between the volume V and the reintroduction device 250 in order to assist this transfer. FIG. 4 schematically and partially shows an example of a device 250 able to allow the purified adipose tissue TP to be reintroduced into the patient's body. The device 250 comprises a body 252 provided with a first port 252a connected to the discharge segment 12 (connection not shown), a second port 252b connected to a syringe 254 and a third port 252c connected to an injection cannula 256. The purified adipose tissue is introduced from the treatment volume V into syringe 254 through the first port 252a. The first port 252a is provided with a check valve so as to allow the purified adipose tissue TP to be injected into the patient through the cannula 256 by pressing the plunger of the syringe 254. The surgeon can act directly on the syringe 254 in order to induce the introduction of material into the patient's body (arrow FT), or in a variant, this action can be automated. In connection with FIGS. 5 to 7, a variant has been shown in which the device further comprises a collapsible element 54 to assist with discharge located around the inlet orifice 110 of the discharge duct 11, said collapsible element being configured to go from a deployed configuration in the purification configuration to a collapsed configuration in response to the winding of the filter in the discharge configuration. FIG. 5 shows the collapsible element 54 in the deployed configuration, FIG. 6 shows the change to the collapsed configuration under the effect of the winding of the filter 5 and FIG. 7 shows the collapsible element 54 in the collapsed configuration. The collapsible element is located in the treatment volume V and is integral with the filter 5. For example, it may be glued to the filter. The expression “comprised between . . . and . . . ” should be understood to include the bounds.

1. A device for purifying adipose tissue and discharging the purified tissue comprising:
a chamber,
a flexible filter delimiting a treatment volume and being able to retain the adipose tissue and to filter a liquid medium, the filter being present in the chamber and extending along a main axis between a lower end and an upper end,
a lower attachment element secured to the lower end of the filter and an upper attachment element secured to the upper end of the filter,
a discharge duct extending into the treatment volume between a lower portion provided with the inlet orifice and an upper portion in communication with the outside of the device, and
a drive system configured to take a purification configuration in which the lower and upper attachment elements are rotated jointly around the main axis and configured to take a discharge configuration in which one of the attachment elements is rotated around the main axis and in relative motion with regard to the other attachment element so as to wind the filter on itself.

2. The device according to claim 1, wherein the device comprises a rigid cage for retaining the filter, surrounding the filter and situated between it and the chamber, which is able to be traversed by the liquid medium.

3. The device according to claim 2, wherein the rigid retention cage defines the upper attachment element and is configured to be rotated in the purification configuration.

4. The device according to claim 1, wherein said other attachment element is fixed in the discharge configuration.

5. The device according to claim 1, wherein the device further comprises a collapsible element to assist discharge located around the inlet orifice of the discharge duct, said collapsible element being configured to go from a deployed configuration in the purification configuration to a collapsed configuration in response to the winding of the filter in the discharge configuration.

6. An assembly for purifying adipose tissue and introducing purified adipose tissue into the body of a patient, the assembly comprising a device, a discharge segment in communication with the upper portion of the discharge duct, and a surgical device comprising (i) a reservoir for receiving purified adipose tissue and in communication with the discharge segment, and (ii) an introduction element in communication with the reservoir and adapted to introduce the purified adipose tissue from the reservoir into the patient's body.

7. A method for purifying adipose tissue using a device containing adipose tissue to be purified in the treatment volume, the method comprising:
centrifuging the adipose tissue by placing a drive system in the purification configuration, and
discharging the purified adipose tissue, obtained after centrifugation, by placing the drive system in the discharge configuration so as to wind the filter on itself and thus apply a discharge pressure to the purified adipose tissue allowing it to be transported outside the device through the discharge duct.

8. The device of claim 1, further comprising:
an adipose tissue filtration element.

9. The device of claim 9, wherein the filtration element further comprises a collapsible element to assist discharge located around the inlet orifice of the discharge duct, said collapsible element being configured to go from the deployed configuration in the purification configuration to the collapsed configuration in response to the winding of the filter in the discharge configuration.

10. The assembly of claim 6, wherein the device comprises:
a chamber,
a flexible filter delimiting a treatment volume and being able to retain the adipose tissue and to filter a liquid medium, the filter being present in the chamber and extending along a main axis between a lower end and an upper end,
a lower attachment element secured to the lower end of the filter and an upper attachment element secured to the upper end of the filter,
a discharge duct extending into the treatment volume between a lower portion provided with the inlet orifice and an upper portion in communication with the outside of the device, and
a drive system configured to take a purification configuration in which the lower and upper attachment elements are rotated jointly around the main axis and configured to take a discharge configuration in which one of the attachment elements is rotated around the main axis and in relative motion with regard to the other attachment element so as to wind the filter on itself.
